(19) United States
(12) Patent Application Publication
(10) Pub. No.: US 2019/0277851 A1
(43) Pub. Date: Sep. 12, 2019

(54) METHODS OF DETECTING CANCER

(71) Applicant: Ascendant Diagnostics, LLC, Springdale, AR (US)

(72) Inventors: Anna DAILY, Fayetteville, AR (US); Lindsay RUTHERFORD, Fayetteville, AR (US)

(21) Appl. No.: 16/356,879

(22) Filed: Mar. 18, 2019

Related U.S. Application Data
(63) Continuation of application No. 14/879,982, filed on Oct. 9, 2015.
Application No. 14/879,982 is a continuation-in-part of application No. 14/707,089, filed on May 8, 2015.
(60) Provisional application No. 62/061,900, filed on Oct. 9, 2014.
(60) Provisional application No. 61/991,061, filed on May 9, 2014.

(51) Int. Cl. G01N 33/574; G01N 30/72; C12Q 1/6886

(52) U.S. Cl. CPC G01N 33/57415; G01N 30/7233; H01J 49/40; G01N 33/57496; G01N 33/57488; C12Q 1/6886

(57) ABSTRACT

Method and kits for detecting cancer, and in particular breast cancer, in a subject by measuring the levels of at least one of a series of biomarkers, as compared to a control sample lacking cancer. The expression of the biomarker either increases or decreases in samples from subjects with cancer, as compared to the expression level in subjects without cancer. The sample is optimally an ocular sample, such as an isolated tear sample or ocular wash, but can also be from saliva, or other bodily fluid. Kits can include a collection tube and protease inhibitors or protein stabilizers.

### CROSS REFERENCE TO RELATED APPLICATIONS

This application claims the benefit of U.S. Provisional Patent Application No. 62/061,900, filed on Oct. 9, 2014, which is hereby incorporated by reference, in its entirety. This application is also related to pending U.S. patent application Ser. No. 14/707,089, filed May 8, 2015, entitled “Methods of Cancer,” which claims priority to U.S. Provisional Application No. 61/991,061 filed on 9 May 2014. The disclosures of each of the above-referenced applications are herein incorporated by reference in their entirety.

### BACKGROUND

The present application encompasses proteins and peptide fragments of those proteins, which are produced by proteolytic digestion of the proteins, and which both proteins and peptide fragments are useful for diagnosing of cancer or for monitoring for the presence of cancer in an individual.

Screening mammograms typically have a sensitivity of 75% and specificity of around 98% resulting in a false positive rate of roughly 5% per mammogram (Brown, Holm, Sickles, & Kessler, 1995; Kolb, Lichy, & Newhouse, 2002; Luftner & Possinger, 2002). Breast tissue type, more specifically density, also greatly influences the performance of mammography. The degree of breast density is classified using the American College of Radiology Breast Imaging Reporting and Data System (BI-RADS). This system consists of four classifications 1-4; where category 1 is mostly fatty (<25% dense); category 2 is scattered fibroglandular densities (25-50% dense); category 3 is heterogeneously dense (51-75% dense) and category 4 is extremely dense (>75% dense) (Bigenwald, 2008; Klifa, 2010; Scheel, 2014).

For women with fatty breast tissue, mammography can be an effective screening tool, when patients are compliant with yearly screenings (Tabar, 2001; Pisano, 2005). However, as breast density increases, the effectiveness of mammography decreases leading to increased follow up imaging and, more importantly, missed cancer diagnosis. Mammographic sensitivity, for high-risk patients, has been shown to be as low as 31% for category 1, 27% for category 2, 20% for category 3, and 12.5% for category 4 (Bigenwald, 2008). Approximately fifty percent of the female population is in categories 3 and 4, which is considered dense breast tissue (Vachon, 2007). Currently, the best screening option for these women is MRI, which can be up to 10 times more expensive than mammography (Beignwald, 2008). Lack of good screening options is a serious problem as women with 75% or more dense tissue have four to six times greater risk of developing breast cancer than women with less dense tissue (Boyd, 2007).

Follow up imaging to evaluate false positives costs the US over 4 billion dollars with an additional 1.6 billion dollars spent for biopsies alone. In 2010 of the 1.6 million biopsies performed, as few as 16% (only 261,000) were found to have cancer (Grady, 2012). The answer to increasing the diagnostic parameters of imaging can be found in the pre- and post-image diagnostics that focus on genetic and proteomic information, more specifically, biomarkers (Armstrong, Handorf, Chen, & Bristol Demeter, 2013; Li, Zhang, Rosenzweig, Wang, & Chan, 2002).

Tissue and serum are commonly the most logical place for beginning biomarker research, however the large dynamic range of both mediums makes discovery quite difficult (Schiess, Wollscheid, & Aebersold, 2009). The answers may lie in less complex biological fluids, such as saliva and tears. The use of tears as diagnostic medium is not a novel application as the tear proteome has been extensively investigated previously (Böhm et al., 2012; 2011; Lebrecht, Boehm, Schmidt, Koelbl, & Grus, 2009a; Lebrecht et al., 2009b; Wu & Zhang, 2007). In this application a quantitative assay for the detection of a panel of tear-based biomarkers in response to cancer is disclosed. From this quantitative information, the framework for a Certified Laboratory Improvement Amendments (CLIA) protocol will be defined.

### SUMMARY

Methods of determining whether a subject has cancer are provided herein. Methods include obtaining a sample from the subject and performing steps of detecting the level of at least one of the markers selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zincα-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3), Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90) Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY). The subject is likely to have cancer if the levels of these markers change in comparison to the levels in a control sample from a subject lacking cancer. The sample is optimally an ocular sample, such as an isolated tear sample or ocular wash, but can also be from saliva, or other bodily fluid. An ocular sample indicates a tear sample encompassing secretions from the lacrimal gland and other tissues that connect with the lymphatic system.

Kits for performing methods described herein are also provided. Kits can contain a sample collection platform, a tube for collection and extraction that can comprise a protease inhibitor or other protein-stabilizing agent, sample extraction reagents and testing apparatus.

### BRIEF DESCRIPTION OF THE DRAWINGS

FIG. 1: ANOVA of spectral intensities for the following proteins identified in Experiment 1: (A) Ig Heavy Chain V-IV region HiL, (B) Ig Heavy Chain region V-III region BRO, (C) Ig Heavy Chain V-III region VH26, (D) Antileukoproteinase, (E) β2 Microglobulin, (F) Calmodulin like protein 5, (G) Lipocalin 1, (H) Cystatin B, (I) Galectin 3, (J) Zinc-α 2 glycoprotein.

FIG. 2: ROC curves for cancer, benign, and control, from Experiment 1, using Ig Heavy Chain V-IV region HiL, Ig Heavy Chain region V-III region BRO, Ig Heavy Chain V-III region VH26, Antileukoproteinase, β2 Microglobulin, Calmodulin like protein 5, Lipocalin 1, Cystatin B, Galectin 3, Zinc-α 2 glycoprotein.

FIG. 3: Mosaic plot of the distribution of breast density for 66 of the 75 samples tested in Experiment 1.

FIG. 4: ANOVA of spectral intensities for proteins identified in Experiment 2. Proteins shown are: (A) 14-3-3 protein sigma (B) 6-phosphogluconate dehydrogenase, decarboxylating (C) Alpha-actinin-4 (D) Retinal Dehydrogenase (E) Argininosuccinate synthase (F) Beta-2-microglobulin (G) Calmodulin (H) Ceruloplasmin (I) Cofilin (J) Cystatin-SN (K) Enolase 1 (L) Gelsolin (M) Heat shock protein beta-1 (N) Ig alpha-1 chain C region (O) Lacritin (P) Lysozyme (Q) Basement membrane-specific heparin sulfate proteoglycan core protein (R) Polymeric immunoglobulin receptor (S) Profilin 1 (T) S100A8 (U) S100A9 (V) Secretoglobin family 1D member 1 (W) VEGF coregulated chemokine 1 (X) Histidine triad nucleotide-binding protein 1 (Y) Definsin 1 (Z) L-lactate dehydrogenase A chain (AA) SPARC-like protein 1 (BB) Annexin 5 (CC) Ig heavy chain V-III region TIL (DD) Inter-alpha-trypsin inhibitor heavy chain 1 (EE) Alpha-1B-glycoprotien (FF) Alpha-1-antitrypsin.

FIG. 5: ANOVA comparing expression levels of S100A9, as determined by ELISA, in breast cancer and control samples.

FIG. 6: ANOVA comparing expression levels of LG3BP, as determined by ELISA, in breast cancer and control samples.

FIG. 7: ROC curve for S100A9

FIG. 8: ROC curve for LG3BP

FIG. 9: ROC curve of S100A9 and LG3BP combined.

FIG. 10: ANOVA for S100A9 expression based on breast tissue category.

FIG. 11: ANOVA of LG3BP expression based on breast tissue category.

### DETAILED DESCRIPTION

Provided herein are proteins and peptide fragments obtained by trypsin digestion from ocular samples. The produced polypeptides are selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zinc-α-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3) Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90), Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY). The trypsin sequences and full-length amino acid sequences of the proteins identified as being down regulated in cancer samples are provided in Appendix I.

The protein and peptides are shown in the Examples to either increase or decrease in biological samples in response to the presence of breast cancer as compared to controls. These proteins and peptides are biomarkers and will be used to determine the disease state of a patient or other subject.

Subjects include humans, domesticated animals such as cats, dogs, cows, pigs, or other animals susceptible to cancer. A “patient” indicates a subject who is diagnosed with a disease, or with cancer, or is being tested for having cancer. Thus the terms subject and patient can be used interchangeably herein. The subjects can be suspected of having cancer. The subject can, be suspected of having cancer, including breast cancer, acoustic neuroma, acute lymphoblastic leukemia, acute myelogenous leukemia, adrenal tumors, AIDS-associated cancers, basal cell carcinoma, benign blood disorders, bladder cancer, bone cancer, brain tumors (metastatic and primary), breast cancer, cancer of unknown primary origin, cervical cancer, chronic lymphocytic leukemia, chronic myelogenous leukemia, colorectal cancer, esophageal cancer, gallbladder and bile duct cancers, gastrointestinal neuroendocrine tumors, GERD, Barrett's esophagus and achalasia, gestational trophoblastic disease, head and neck cancers, Kaposi sarcoma, kidney cancer, leukemias, liver cancer, liver metastases, low-grade glioma, lung cancer, lymphoma, male breast cancer, melanoma, Merkel cell carcinoma, mesothelioma, multiple myeloma, myelodysplastic syndrome, ovarian cancer, pancreatic cancer, pancreatic cysts, pituitary tumors, prostate cancer, pulmonary neuroendocrine tumors, rare blood disorder, skin cancer, soft tissue sarcoma, spine tumors, squamous cell carcinoma, stomach (gastric) cancer, testicular cancer (germ cell tumors), thymoma and other thymic tumors, tracheal diseases, uterine (endometrial) cancer, uterine sarcoma. The subjects can have an increased risk of developing breast cancer. For example, the subject can be at increased risk of cancer, or suspected of having cancer because of a positive mammography result, by detection of a lump in the breast, testing positive for a gene known to increase the risk of cancer such as BRCA, or already had a resection, biopsy, or other procedure to remove the cancer. The subject can be undergoing, or have previously undergone, treatment for cancer and methods and kits herein are used to monitor progression of treatment or alternatively to monitor for recurrence or spread of the cancer.

Also provided herein are methods and kits to collect ocular samples for use in determining the expression levels of the identified proteins or polypeptides in lacrimal secretions. The use of collection strips and tubes containing protease inhibitor or protein stabilizing agents is disclosed. Kits further contain buffers or reagents for the elution of breast cancer biomarkers from the collection strips that have been in contact with an eye to collect lacrimal secretions. The design of devices to collect proteins from the ocular cavity, as well as the packaging of this device with a container to house the collection device and elution buffers, is disclosed.

Methods disclosed herein encompass the use of these cancer biomarkers, singly or in multiples, in a CLIA based protocol utilizing a triple quadrupole LC-MS/MS platform, which will be carried out at a centralized laboratory testing facility. The ocular samples collected from individuals can be shipped to the testing facility in this embodiment. The identified proteins and their subsequent proteolytic fragments, as shown in Appendix I, are used for quantitative analysis of diagnostic peptides produced in the triple quad. A threshold value or a relative or actual value in terms of polypeptide concentration directly relating to the polypeptides selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zinc-α-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3), Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALK), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90), Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY) can be defined or samples can be compared directly to non-cancerous controls. The quantitative information in report form can be provided to physicians to help in making decisions regarding the pathway of patient care. Physicians can base treatment decisions on these results and the final step can include administration of an appropriate anti-cancer therapeutic to the subject.

In an alternate embodiment, the polypeptides selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zinc-α-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1, (PROF1), Cluster of Ig lambda chain V-III region LOI (LV302), Prothrombin (E9PIT3), Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90), Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY) can be detected by implementing binding agents, for example antibodies, peptoids, or coated surfaces, and reagents that accommodate a binding interaction specific to these proteins to produce a reaction that can be quantitated based on production of a detectable signal such as florescence, color change, or UV absorbance. Implementing these components in a cartridge with a partnering reading instrument, such as a point-of-care device that can be used at point of care is also provided. Binding agents for these proteins and polypeptides can also be used for detection in a lateral flow device. Thus, methods of detecting the level of protein expression in the samples using a binding partner such as an antibody can be used to detect the markers provided herein in an immunoassay.

The immunoassay typically includes contacting a test sample with an antibody or antigen that specifically binds to, or otherwise recognizes a biomarker, and detecting the presence of a complex of the antibody or antigen bound to the biomarker in the sample. The immunoassay procedure can be selected from a wide variety of immunoassay procedures known in the art involving recognition of antibody/antigen complexes, including enzyme-linked immunosorbent assays (ELISA), radioimmunoassay (RIA), and Western blots, and use of multiplex assays, including use of antibody arrays, wherein several desired antibodies are placed on a support, such as a glass bead or plate, and reacted or otherwise contacted with the test sample. Such assays are well-known to the skilled artisan.

The detection of the biomarkers described herein in a sample can be performed in a variety of ways. In one embodiment, the method provides the reverse-transcription of complementary DNAs from mRNAs obtained from the sample. Fluorescent dye-labeled complementary RNAs can be transcribed from complementary DNAs that are then hybridized to the arrays of oligonucleotide probes. The fluorescent color generated by hybridization can be read by machine, such as a SureScan microarray scanner (Agilent Technologies) and data obtained and processed using software, such as Agilent Feature Extraction Software (9.1). Such array-based methods include microarray analysis to develop a gene expression profile. As used herein, the term “gene expression profile” refers to the expression levels of mRNAs or proteins of a panel of genes in the subject. As used herein, the term “diagnostic panel” refers to a panel of genes, peptides or proteins with an expression level that can be relied on to diagnose or predict the status of the disease. Included in this panel are genes, peptides and proteins selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zinc-α-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3), Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90) Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY) as well as any combination thereof, as provided herein.

In other embodiments, complementary DNAs are reverse-transcribed from mRNAs obtained from the sample, amplified, and simultaneously quantified by real-time PCR, thereby enabling both detection and quantification (as absolute number of copies or relative amount when normalized to DNA input or additional normalizing genes) of a specific gene product in the complementary DNA sample as well as the original mRNA sample.

The methods of the versions of this invention include detecting at least one biomarker. However, any number of biomarkers can be detected. It is preferred that at least two biomarkers are detected in the analysis. However, it is realized that three, four, or more, including all, of the biomarkers described herein can be utilized in the analysis. Thus, not only can one or more markers be detected, any number or combination of markers can be used in detection. In addition, other biomarkers not herein described can be combined with any of the presently disclosed biomarkers to aid in the diagnosis of cancer. Moreover, any combination of the above biomarkers can be detected in accordance with versions of the present invention.

The markers selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zinc-α-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (ALIA1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3), Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondria! (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90), Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY) can increase or decrease at least 1.5 fold, 2 fold, 4 fold, 5 fold, 8 fold, 10 fold or more, relative to the level of the marker in the control sample. The control sample can be a sample from a subject that does not have cancer, a pooled sample from subjects that do not have cancer, or can be a control or baseline expression level known to be the average expression level of subjects without cancer.

Several terms are used throughout this disclosure and should be defined as commonly used in the art, or as specifically provided herein. As provided herein, mass spectrometry or MS refers to an analytical technique generating electrical or magnetic fields to determine mass-to-charge ratio of peptides and chemical compounds in order to identify or determine peptide sequence and chemical structures. LC-MS/MS spectrometry refers to an analytical technique combining the separation capabilities of high performance liquid chromatography (HPLC) with the mass analysis of mass spectrometry. Triple quadrupole mass spectrometry refers to a tandem mass spectrometer with three ionizing chambers (Q1, Q2, & Q3). This technique allows for target detection of molecules of interest. Ion pairs refers to a parent peptide detected in Q1 in its doubly or triply charged form and a resulting y or b ion as generated by Q2 and detected in Q3 of a triple quadrupole mass spectrometry instrument. SIS internal peptide refers to a synthesized isotopically-labeled peptide with the same sequence as the peptide to be monitored in Q1 and used as an internal standard for reference to quantify the peptide of interest. The −y ion refers to an ion generated from the c-terminal of a peptide fragment. The −b ion refers to an ion generated from the n-terminal of a peptide fragment. Quantitative Ion refers to the selected highest intensity y or b ion used to determine the quantity of its parent protein in a biological sample. Qualitative Ion refers to ion/ions chosen to ensure the integrity of the Qualitative Ion to selected protein of interest and labeled peptide to selected standards.

CLIA refers to Clinical Laboratory Improvements Amendments, which are federal regulatory standards that apply to all clinical laboratory testing preformed on humans in the United States, except clinical trials and basic research. (CLIA related Federal Register and Code of Federal Regulation Announcements). CLIA approved laboratory refers to a clinical lab that preforms laboratory testing on human specimens for diagnosis, prevention, or treatment of disease or impairment and is approved and monitored by a Food and Drug Administration (FDA) approved regulatory organization (CLIA Laws and Regulations, 2013). CLIA-waived test refers to a clinical laboratory test meeting specific criteria for risk, error, and complexity as defined by the FDA.

Point-of-care device refers to an instrument or cartridge available at the location of patient and physician care, which contains binding agents to a biomarker, or series of biomarkers of interest, and which can generate information on the presence, absence, and in some cases concentrations of detected biomarkers. Analyte refers to any measurable biomarker, which can be protein, peptide, macromolecule, metabolite, small molecule, or autoantibody. Biological fluid, as used herein, refers to tears, whole blood, serum, urine, and saliva. Biomarker refers to any substance (e.g. protein, peptide, metabolite, polynucleotide sequence) the concentration level of which changes in the body, for example increased or decreased, as a result of a disease or condition. Marker and biomarker can be used interchangeably used herein.

Lateral flow test refers to a device that measures the presence of an analyte in a biological fluid using porous paper of sintered polymer. ELISA refers to Enzyme-linked immunosorbent assay, which utilizes antibodies or antigens to detect the presence and concentration of an analyte of interest. Diagnostic panel refers to a group of molecules for example proteins or peptides, the combined concentrations of which are used to diagnose a disease state, for example cancer. A breast cancer marker refers to a molecule: for example protein, peptide, metabolite, polynucleotide sequence, the concentration level of which changes in the body: for example increased or decreased, as a result of the presence or absence of cancer.

In addition to being useful to diagnose cancer, and in particular breast cancer, in a subject, kits and methods provided herein can be used to monitor treatment or recurrence of cancer in an individual previously diagnosed with cancer. Thus if the levels of the markers selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zinc-α-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADH1G), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3), Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90) Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY) increases or decreases over time in the same subject after treatment, further chemotherapeutics targeting the cancer can be administered.

Methods and kits can also be used to monitor the effectiveness of a chemotherapeutic treatment. In this alternate embodiment, the levels of the biomarkers selected from the following and as listed in Table 1: Ig lambda chain V-IV region Hil (LV403), Ig heavy chain V-III BRO (HV305), Ig heavy chain V-III VH26 (HV303), β-2-microglobulin (B2MG), Lipocalin-1 (LCN1), Zinc-α-2-glycoprotein (ZA2G), Cystatin B (CYTB), Antileukoproteinase (SLP1), Galectin-3 (LEG3), Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5 (IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3), Hemopexin (HEMO), Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90), Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY) can increase or decrease over time if the treatment regime is effective and either would not change or can increase or decrease over time if the treatment regime is not effective in a single subject, depending on which biomarker(s) are being examined in the subject.

Treating cancer includes, but is not limited to, reducing the number of cancer cells or the size of a tumor or mass in the subject, reducing progression of a cancer to a less aggressive form, reducing proliferation of cancer cells or reducing the speed of tumor growth, killing of cancer cells, reducing metastasis of cancer cells or reducing the likelihood of recurrence of a cancer in a subject. Treating a subject, as used herein, refers to any type of treatment that imparts a benefit to a subject afflicted with a disease or at risk of developing the disease, including improvement in the condition of the subject for example in one or more symptoms, delay in the progression of the disease, delay in the onset of symptoms, or delay in the progression of symptoms, etc.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language for example “such as,” provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values near to the recited amount are included in that amount, such as values that can or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what that author asserts, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references. Any references mentioned are not admitted to be prior art with respect to the present invention.

The following examples are meant only to be illustrative and are not meant as limitations on the scope embodiments of the invention or of the appended claims.

### EXAMPLES

### Example 1

### Participant Selection

Study participants were recruited from patients being seen at Breast Health and Surgery Centers AR, OK, TN and WA under Institutional Review Board approval. All patients were consented and provided a copy of the informed consent prior to sample collection or completing patient information sheet. Samples were collected by clinic staff (i.e. nurses and technicians) and/or Ascendant Dx staff. Inclusion/exclusion criteria used for patient selection were as follows:

### Inclusion Criteria:

Individuals between the ages of 18-100 years of age,
Presenting for a routine check-up, or
Presenting for the evaluation of an abnormal exam or test, or
Presenting for the evaluation of palpable mass, or
Presenting with a mass pre or post biopsy as long as a portion of the mass is remaining, or
Have recently been diagnosed with breast cancer but have not undergone treatment of any kind.

### Exclusion Criteria:

Individuals below the age of 18 or over 100
Experiencing a concurrent eye infection or trauma, or
Currently experiencing acute conjunctivitis, or
Have been diagnosed with breast cancer and have undergone treatment.

Control samples were collected from patients being seen for routine screening mammograms and did not receive a call back for additional procedures. Benign samples were collected at the time of biopsy and were included in the benign group once the pathology results were determined. Cancer samples were also collected at time of biopsy and included in the cancer group after pathology results were known, from patients having MRI's prior to surgery, and from patients undergoing sentinel node procedures prior to surgery.

Data collected from participants included the following: age, sex, race, currently taking birth control or on hormone replacement therapy, ophthalmological infections, current or recent chemotherapy treatments, family history of cancer, genetic testing (BRAC1/2) if available, cancer stage (I, II, III, IV), cancer type (Ductal Carcinoma In Situ, Invasive Ductal Carcinoma, invasive Lobular Carcinoma, Lobular Carcinoma In Situ, and Unknown), hormone receptor status (ER+/−, PR+/−, HER2+/−), size of mass, tumor grade (I, II, III), breast density score (densities identified as category 1, 2, 3, or 4) and previous history of cancer.

### Example 2

### Schirmer Strip Sample Collection Procedure

Institutional review board approval was obtained for the collection of tears using Schirmer strips (GuldenOpthalmics Elkins Park, Pa.). For collection, the rounded tip of the Schirmer strip was folded over at the 0 mm line forming a lip. The folded portion was placed in the lower eyelid of the participant and they were asked to close their eye and keep it in the closed position for a period of 5 minutes. After five minutes the strip was removed and placed in a sterile 1.5 mL pre-labeled snap top tube and placed at −20° C. or −80° C. depending on availability. Collection criteria stated that if the 35 mm mark was reached prior to the five minute time, the strip can be removed. Samples collected at participating clinics were retrieved by Ascendant Dx staff on a weekly basis and transferred on dry ice to Ascendant Dx's laboratory facility.

### Example 3

### Schirmer Strip Sample Processing

Protein elution was carried out by first dicing the strips, using sterile tweezers and scissors, into clean sterile 1.5 mL snap top tube. 200 μL of 1× Phosphate-buffered saline (PBS) was added to the diced strip and the sample was incubated at 4° C. with mild shaking for 3 hours. Following elution, the samples were spun briefly in a tabletop centrifuge to collect the strip fragments at the bottom of the tube, and the supernatant was transferred to a new clean 1.5 mL snap top tube. Total protein content was determined using standard bicinchoninic acid (BCA) assay, and the samples were stored at −80° C. until further use.

Total protein content of each pool was determined using a BCA assay kit (Pierce) with a 1:20 (v/v) ratio of standard and unknown to working reagent and an incubation time of 30 min at 37° C. To ensure reliable total protein content calculation, a series of dilutions were made for each sample, for example 1:2, 1:4, 1:6, and all dilutions were plated in triplicate. A standard curve using diluted albumin (2 mg/ml, 1.5 mg/ml, 1 m/ml, 0.75 mg/ml, 0.5 mg/ml, 0.25 mg/ml 0.125 mg/ml 0.025 mg/ml and 0 mg/ml) was generated and blank subtraction was applied to all standards and unknowns. The protein concentration for each unknown was calculated using a four-parameter fit of the standard curve. Concentrations were multiplied by the dilution factor and averaged to give an accurate total protein content calculation. Assays were considered valid if the coefficient of variation (% CV) was 15% or below.

### Example 4

### Methods and Results for Label Free Quantitation by LC MS/MS

Experiment 1: In solution trypsin digestion followed by LC MS/MS was carried out on 25 breast cancer samples, 25 benign samples, and 25 control samples by the Proteomic Core at the University of Arkansas for Medical Sciences (UAMS). Solution digests were carried out on all 75 samples in 100 mM ammonium bicarbonate (Sigma-Aldrich), following reduction in 10 mM Tris[2-carboxyethyl]phosphine (Pierce) and alkylation in 50 mM iodoacetamide (Sigma-Aldrich), by addition of 100 ng porcine trypsin (Promega) and incubation at 37° C. for 12-16 hours. Peptide products were then acidified in 0.1% formic acid (Fluka). Tryptic peptides were separated by reverse phase Jupiter Proteo resin (Phenomenex) on a 100×0.075 mm column using a nanoAcquity UPLC system (Waters). Peptides were eluted using an 80 min gradient from 97:3 to 35:65 buffer A:B ratio. [Buffer A=0.1% formic acid, 0.05% acetonitrile; buffer B=0.1% formic acid, 75% acetonitrile.] Eluted peptides were ionized by electrospray (1.8 kV) followed by MS/MS analysis using collision-induced dissociation on an LTQ Orbitrap Velos mass spectrometer (Thermo). MS data were acquired using the FTMS analyzer in profile mode at a resolution of 60,000 over a range of 375 to 1500 m/z. MS/MS data were acquired for the top 15 peaks from each MS scan using the ion trap analyzer in centroid mode and normal mass range with a normalized collision energy of 35.0. Proteins were identified from MS/MS spectra by database searching the Mascot search engine (Matrix Science) or MaxQuant quantitative proteomics software (Max Planck Institute). Mascot search results were compiled using Scaffold (Proteome Software).

The following criteria were set to select a group of proteins that can be indicative of altered breast physiology: 1) protein has a fold change of 1.5 or greater (in either positive or negative direction with respect to cancer). 2) fold change should be accompanied by p value of <0.05. 3) protein is present in 12 out of the 25 cancer samples. Using these criteria, the list of over 500 was reduced to the following proteins: Alpha-1-antitrypsin (A1AT), Antileukoproteinase (SLP1), Cofilin (COF1), Antithrombin-III (ANT3), Beta-2-microglobulin (B2MG), Protein-glutamine-gamma-glutamyltransferase (B4D1T7), Uncharacterized protein (B8ZZQ6), Calmodulin-like protein 5 (CALL5), Cystatin-B (CYTB), Neutrophil defensing-1 (DEF1), Destrin (DEST), Kaliocin-1 (E7ER44), Cluster of Rab GDP dissociation inhibitor beta (E7EU23), Elongation factor 1-alpha (EF1A1), Ezrin (EZRI), Heme-binding protein 2 (HEB2), Heat Shock cognate 71 (HSP7C), Heat shock protein beta 1 (HSPB1), Ig Heavy chain V-III (HV303), Cluster of Ig Heavy chain V-III region BRO (HV305), Lipocalin-1 (LCN1), Galectin-3 (LEG3), Ig lambda chain V-IV (LV403), Isoform 2 of Ig mu chain c region (P01871), Cluster of isoform 2 heat shock protein (P07900-2), Cluster of 14-3-3 protein zeta delta (P63104), Isoform 3 of Perilipin-3 (PLIN3), Proteasome activator complex subunit 1 (Q06323), UMP-CMP kinase (Q5T0D2), Protein S100-A4 (S10A4), S100-A8 (S10A8), Protein S100-A9 (S10A9), Protein S100-A11 (S10AB), Submaxillary gland androgen-regulated protein (SMR3B), Zinc-alpha-2-glycoprotein (ZA2G), Zymogen granule protein 16 homolog B (ZG16B)

Experiment 2: In solution trypsin digestion followed by LC MS/MS was repeated using fifty samples in each group (breast cancer, benign, control). Identical methods as described for Experiment 1 were used for Experiment 2. The increase in samples size caused some of the trends observed in Experiment 1 to disappear, while making trends emerging in Experiment 1 more prominent in Experiment 2. From this data the list of 500 proteins described in Experiment 1 was further refined down to 103 proteins of interest. This list includes: Histidine triad nucleotide-binding protein 1 (D6RD60), S100A9 (S10A9), S100A8 (S10A8), Galectin-3-binding protein (LG3BP), Cluster of Ig alpha-1 chain C region (IGHA1), Cluster of Ig kappa chain V-III region HAH (KV312), VEGF co-regulated chemokine 1 (VCC1), L-lactate dehydrogenase A chain (LDHA), Aldo-keto reductase family 1 member C (AKR1C1), Rootletin (B1AKD8), L-lactate dehydrogenase B chain (LDHB), Retinal dehydrogenase 1 (AL1A1), Uncharacterized Protein (B4E1Z4), Alpha-1-antichymotrypsin (AACT), Superoxide dismutase [Cu—Zn] (SODC), SPARC-like protein 1 (SPRL1), Ig heavy chain V-III region TIL (HV304), Keratin (K1C9), Cystatin-SN (CYTN), Alpha-actinin-4 (ACTN4), Ig lambda-3 chain C regions (Fragment) (IGLC3), Immunoglobulin lambda-like polypeptide 5(IGLL5), Alcohol dehydrogenase 1C (ADHIG), Malate dehydrogenase, mitochondrial (MDHM), Calmodulin-like protein 5 (CALL5), Alpha-1-antitrypsin (A1AT), Alpha-1B-glycoprotein (A1BG), Leucine-rich alpha-2-glycoprotein (A2GL), Small ubiquitin-related modifier 3 (A8MU27), Anterior gradient protein 2 homolog (AGR2), Profilin-1 (PROF1), Cluster of Ig lambda chain V-III region LOI (LV302), Prothrombin (E9PIT3), Hemopexin (HEMO) Ig gamma-2 chain C region (IGHG2), Ubiquitin-40S ribosomal protein S27a (RPS27A), Afamin (AFAM), Apolipoprotein A-I (APOA1), Apolipoprotein A-IV (APOA4), Flavin reductase (NADPH)(BLVRB), Prosaposin (PSAP), Lacritin (LACRT), 60S acidic ribosomal protein P1 (RLA1), Inter-alpha-trypsin inhibitor heavy chain H2 (ITIH2), Mucin-like protein 1 (MUCL1), S100 A6 (S100A6), Na(+)/H(+) exchange regulatory cofactor NHE-RF1 (NHRF1), Thioredoxin domain-containing protein 17 (I3L0K2), Lymphocyte-specific protein (LSP1), Cluster of Haptoglobin (H3BS21), Myosin regulatory light chain 12A (J2QRS3), Ribonuclease inhibitor (RINI), Alpha-enolase (ENOA), Cluster of Ig kappa chain V-I region EU (KV106), Alcohol dehydrogenase class 4 mu/sigma chain (ADH7), Protein AMBP (AMBP), Angiotensinogen (ANGT), Antithrombin-III (ANT3), Apolipoprotein A-II (APOA2), Calpastatin (B7Z574), Brain acid soluble protein 1 (BASP1), Alpha-2-HS-glycoprotein (C9JV77), Calreticulin (CALR), Calpain-1 catalytic subunit (CAN1), Cell division control protein 42 homolog (CDC42), Complement C3 (CO3), Coronin-1A (COR1A), Programmed cell death 6-interacting protein (DCD), Definsin 1 (DEF1), F-box only protein 50 (FBX50), Gamma-glutamylcyclotransferase (GGCT), Glutathione reductase, mitochondrial (GSHR), Keratin, type II cytoskeletal 1 (K2C1), UMP-CMP kinase (KCY), Mesothelin (MSLN), N-acetylmuramoyl-L-alanine amidase (PGRP2), Nicotinate phosphoribosyltransferase (PNCB), Inter-alpha-trypsin inhibitor heavy chain H1 (ITIH1), Ribonuclease T2 (RNASET2), Superoxide dismutase [Mn], mitochondrial (SODM), Small proline-rich protein 3 (SPRR3), Src substrate cortactin (SRC8), Cluster of Tubulin beta-4B chain (TBB4B), Tropomyosin alpha-3 chain (TPM3), Serotransferrin (TRFE), Glutathione S-transferase P (THIO), Vitronectin (VTNC), Vitamin D Binding protein (Q6LDC6), Inter-alpha-trypsin inhibitor heavy chain H4 (ITIH4), Metalloprotease inhibitor (TIMP1), Heat Shock protein 90 (HSP90), Cathepsin B (CATB), Ceruloplasmin (CERU), Calprotectin, 14-3-3 sigma (1433S), alpha-2-hs-glycoprotein (FETUA), alpha-2-macroglobulin (A2MG), Transthyretin (TTHY) (Appendix 1). The complete list of proteins identified was exported from Scaffold software for analysis using JMPpro11 statistical software package.

### Example 5

### Statistical Analysis using JMPpro11 Software

Experiment 1: The data was organized by using Microsoft Excel® spreadsheet software with spectral intensities of each of the previously identified proteins for all 75 samples. Also included in the spreadsheet were sample status (cancer, benign, control) cancer type, tumor size grouping (1=0.1 mm-1 cm, 2=1.1-2.9 cm, 3=3 cm or greater) and tumor grade status. This spreadsheet was uploaded into the JMPpro11 software package for analysis.

Binary linear regression was applied between cancer and control samples. Six proteins were identified as significant based on their sigmoidal curve and p-value, they were Ig lambda chain V-IV (LV403), Ig heavy chain V-III region BRO (HV305), Ig heavy chain V-III VH26 (HV303), Lipocallin-1 (LCN1), Beta-2-microglobulin (B2MG), Zinc-alpha-2-glycoprotein (ZA2G). All six proteins appeared to be down-regulated with respect to cancer. To determine if the down regulation of these proteins was specific to breast cancer a binary linear regression was repeated between benign and control. This comparison indicated the six previously identified proteins were also down-regulated in benign samples and an additional four proteins were identified as significant between benign and control; Antileukoproteinase (SLP1), Calmodulin-like-protein 5 (CALL5), Cystatin B (CYTB), and Galectin-3 (LEG3).

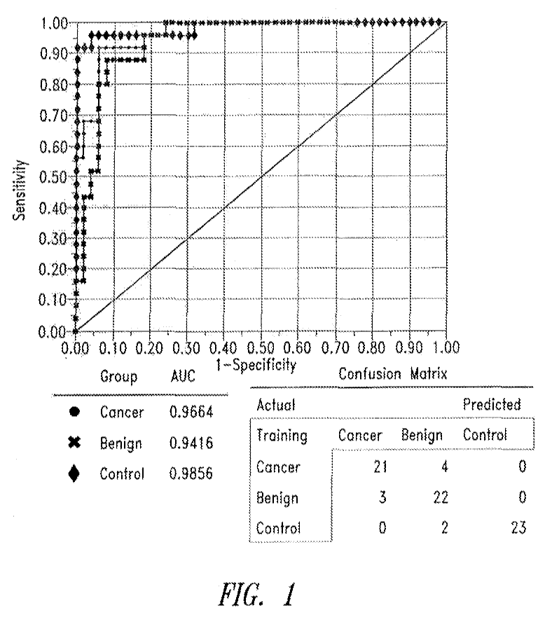

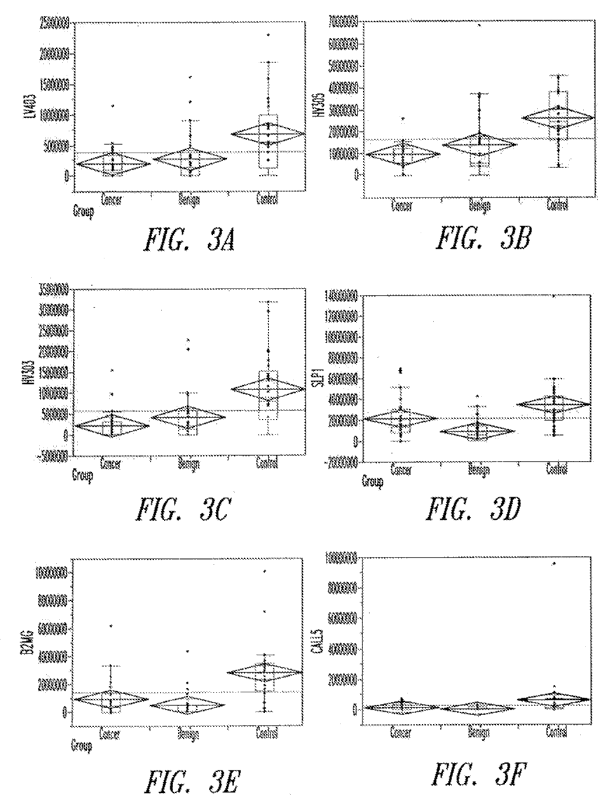

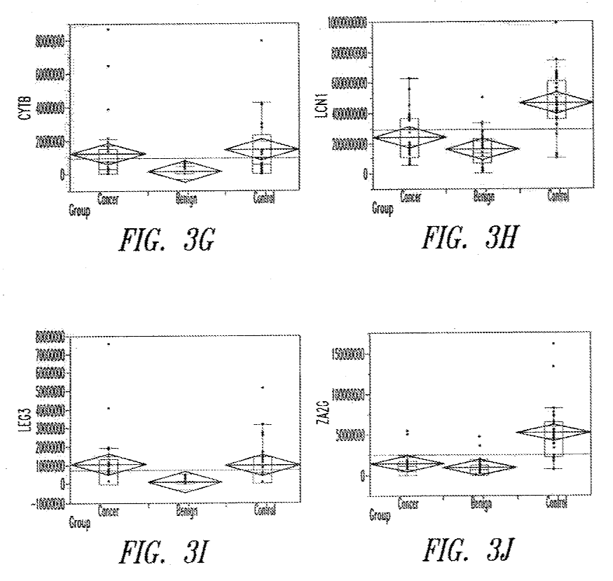

Nominal logistic regression was carried out using all three groups and all ten proteins to determine degree of differentiation between all groups. Degree of misclassification was for the group of ten proteins was 12%. Receiver operator curves for each category produced AUC values of 0.96 for cancer, 0.94 for benign, and 0.98 for control (FIG. 1). One way ANOVA analysis was preformed to ensure a shifting mean value for the three categories of samples by each protein (FIG. 3).

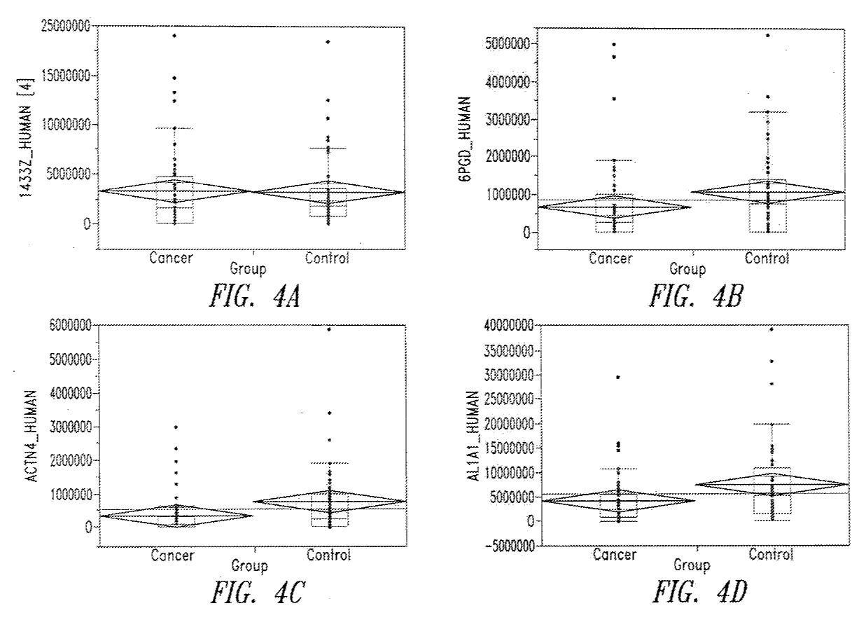

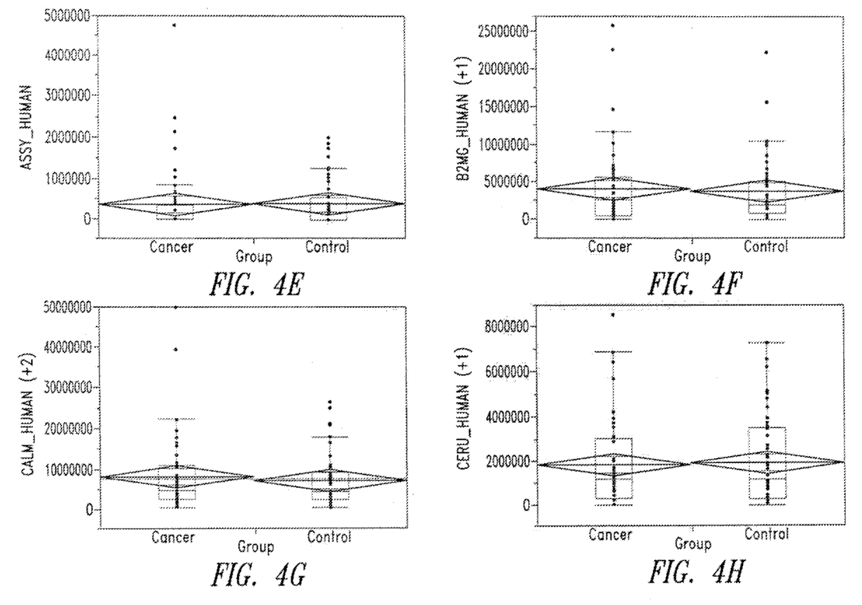

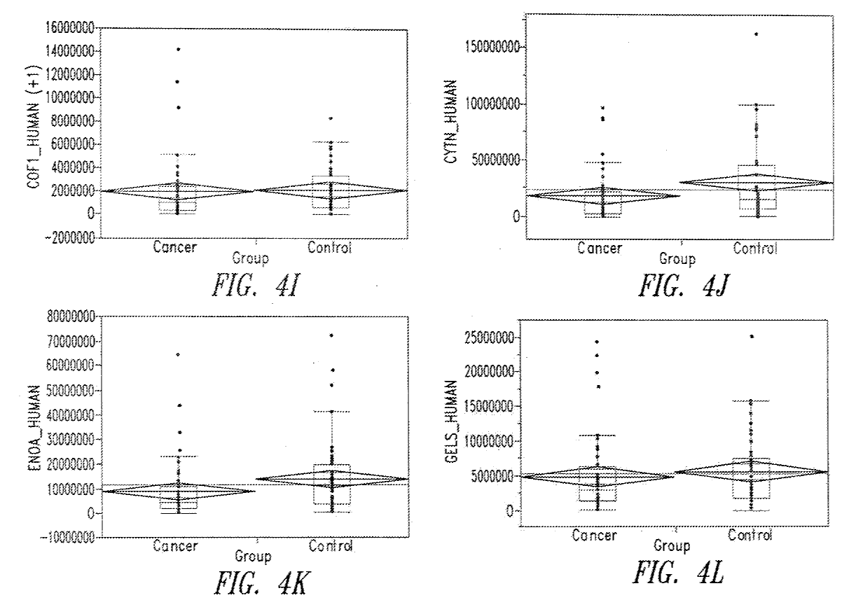

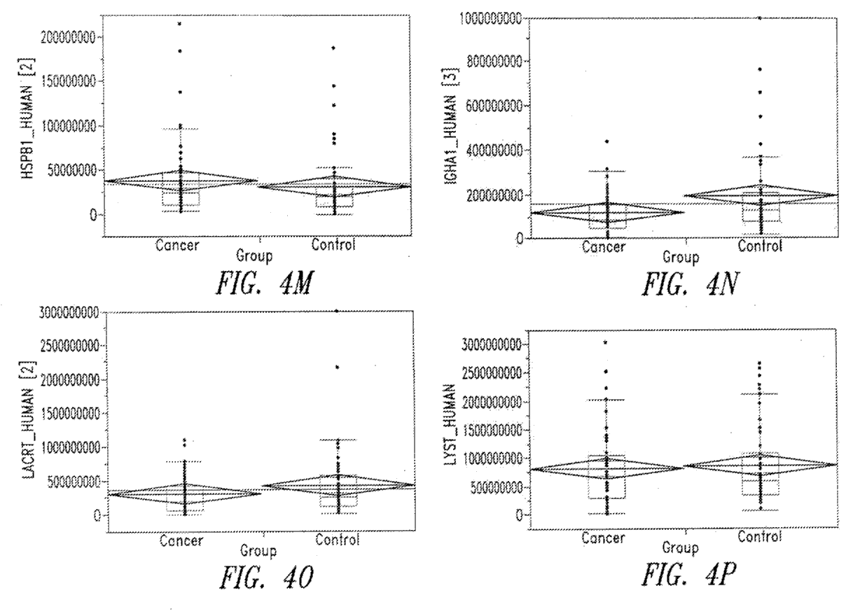

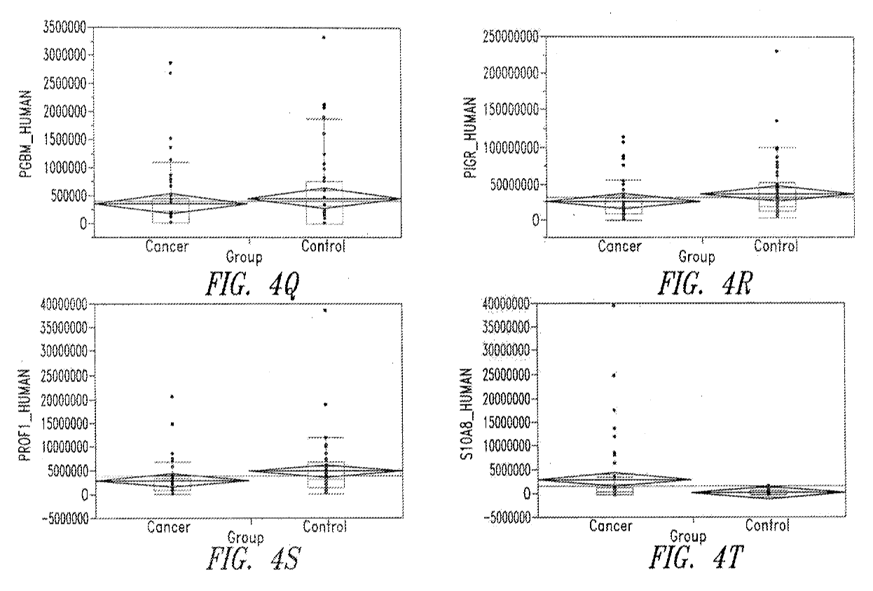

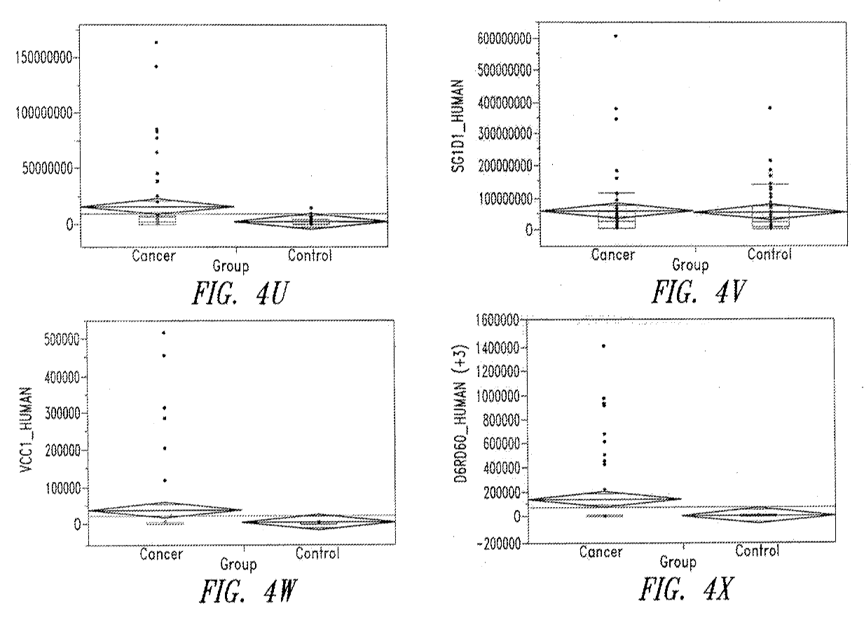

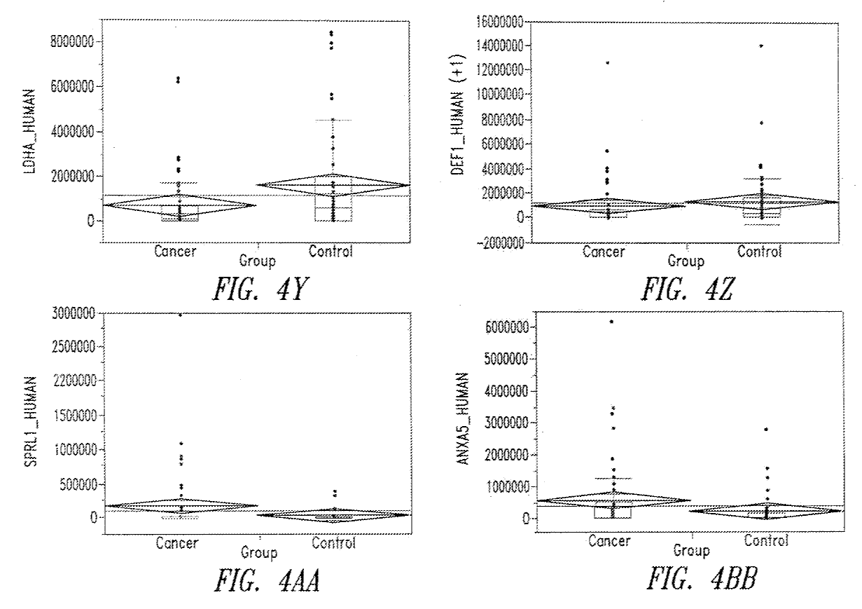

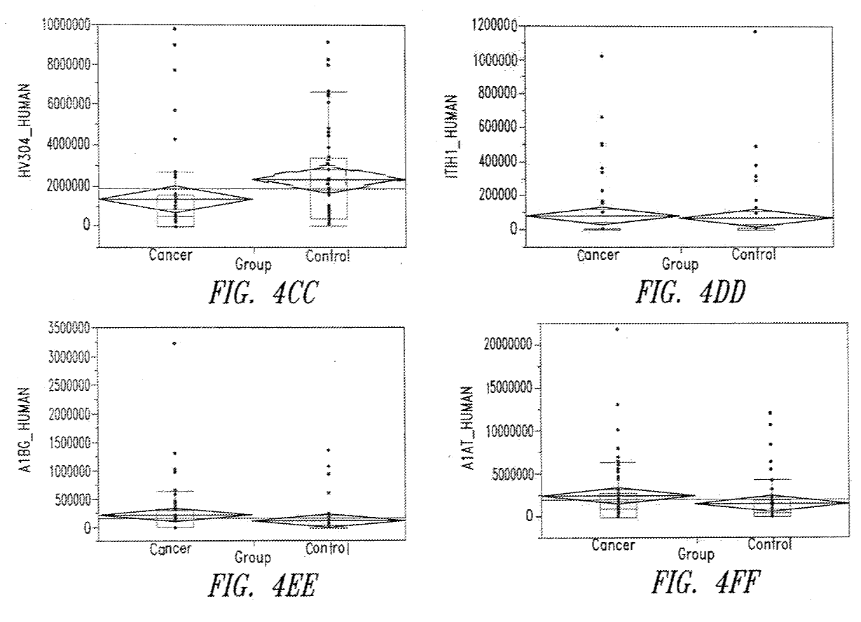

Experiment 2: One-way ANOVA analysis was preformed using spectral intensities recorded for all proteins identified from mass spectrometry. Tryptic peptide products were analyzed for all proteins. Proteins were compared in three ways: 1) control vs cancer, 2) cancer vs benign, 3) benign vs control. An alpha level of 0.05 was used as an indicator of significant expression change between the groups. Variations in spectral intensities of tryptic fragments were compared. A series of proteins was identified where expression level increased in breast cancer samples with respect to control. Proteins were also identified where expression decreased in breast cancer samples with respect to control. To ensure experimental validity, it was established that proteins were present in whose expression remained constant across all groups tested (FIG. 4).

The initial list of 500 proteins was reduced to 103 proteins of interest. Within this group of proteins are proteins with increased expression with respect to breast cancer as well as proteins with decreased expression with respect to breast cancer (FIG. 4). The diagnostic capability of whole protein sequence, tryptic peptides, and sequence anomalies, which can be present as a result of disease, are all of interest.

### Example 6

### ELISA Analysis

A series of proteins with an increase in expression level in cancer samples relative to control samples were observed by mass spectrometry, and several candidates were selected for further validated using ELISA. S100A9 was selected as a representative example of increased expression in tears of breast cancer patients relative to control. LG3BP is shown as a representative of proteins with a decreased expression level in breast cancer samples relative to control samples. Standard ELISA protocol was used to evaluate the expression level of in tears. Data for S100A9 and LG3BP was obtained using kits purchased as DuoSets from R&D Systems (Minneapolis, Minn.). ELISA procedures were carried out according to instructions provided by R&D systems. Briefly, 100 μL per well of capture antibody, diluted to the recommended working concentration (GAL3BP 1.0 ug/ml; S100A9 0.5 ug/ml) in 1×PBS, was added to a high binding 96 well plate and incubated overnight (˜16 hrs) at room temperature. The plate was rinsed with 1×PBS with 0.05% Tween four times, and blotted against clean absorbent paper to remove any traces of liquid following each rinse. Rinse cycles were carried out after each step of the assay. Blocking buffer (1×PBS with 1% BSA) was added to all wells (˜200 μL/well) and the plates were incubated for two hours at room temperature. Tear samples were diluted using 1×PBS with 1% BSA at dilutions of 1:10 and 1:50 for S100A9 and 1:100 & 1:500 for Galectin 3 Binding Protein. Dilutions known to fall within the linear region of the standard curve for each assay were selected based on results from previous optimization assays. Samples and standards were tested in duplicate using 100 μL/well and incubated at room temperature for 2 hours. Detection antibody was diluted to the recommended working concentration (GAL3BP 2 ug/ml and S100A9 1 ug/ml) 100 μL was added to each well, and the plate was allowed to incubate for 2 hours at room temperature. Streptavidin-HRP solution was added to each well (100 μL/well) and allowed to incubate for 15 min at room temperature. Visualization was achieved by addition of TMB substrate solution. After 15 minutes, 50 μL of 1M H₂SO₄ was added to each well, and the absorbance at 450 nm was determined. ELISA data was analyzed using Prism version 6.0 available from GraphPad.

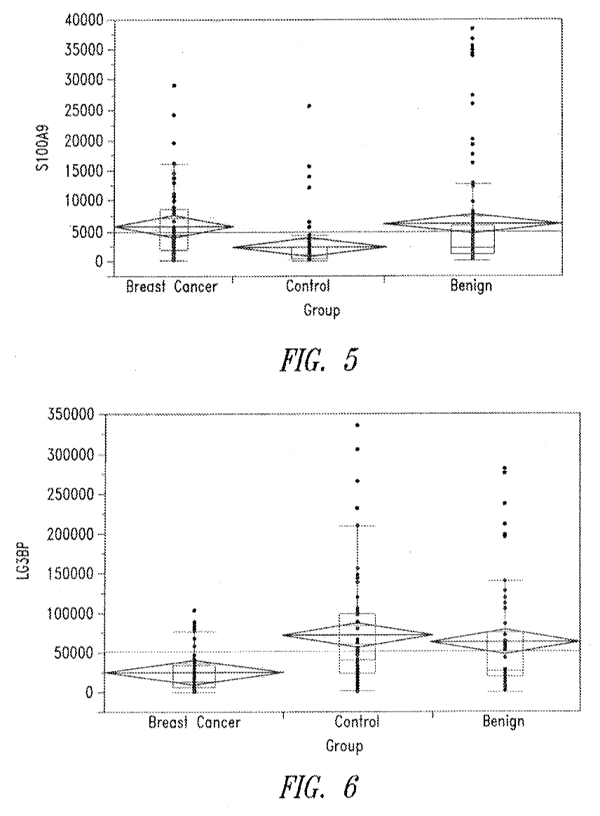

Statistical analysis of ELISA data: Concentrations of each protein, as calculated by Prism software, were exported into JMP Pro11 for statistical evaluation. Numerous candidates were selected to be investigated using ELISA assays on Control, Cancer or Benign samples. Results of two proteins (S100A9 and Galectin-3-Binding Protein) are shown (FIG. 5 and FIG. 6, respectively) to provide representative example of increased and decreased protein expression in breast cancer tear samples with respect to control. ANOVA of S100A9 for 63 breast cancer samples, 79 control samples, and 92 benign samples resulted in a p-value of 0.0005 when all three groups were evaluated. Group means for S100A9 were: breast cancer=5673.02 pg/ml; control=2130.18 pg/ml; benign=6179.10 pg/ml. S100A9 expression is increased by 2.6 fold in cancer samples compared to control samples and 2.9 fold increase in benign samples compared to control. ANOVA of Galectin-3 Binding Protein for 66 breast cancer samples, 55 control samples, and 54 benign samples resulted in a p-value of <0.0001 when all three groups were evaluated. Group means for LG3BP were: breast cancer=24448.1 pg/ml; control=70242.2 pg/ml; benign 62329.7 pg/ml. LG3BP has a 2.8 fold increase in control samples compared to cancer samples and a 2.5 fold increase in benign samples compared to cancer.

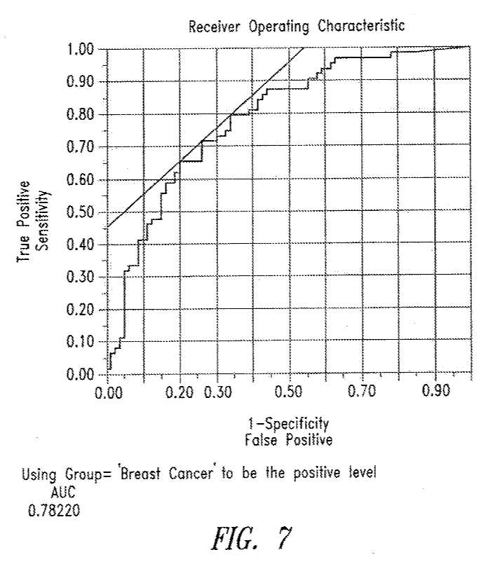

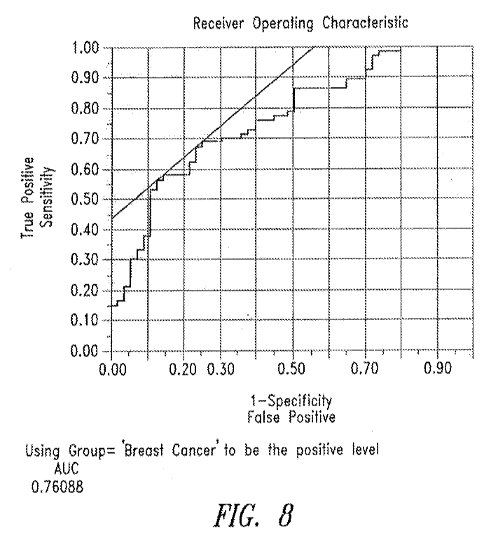

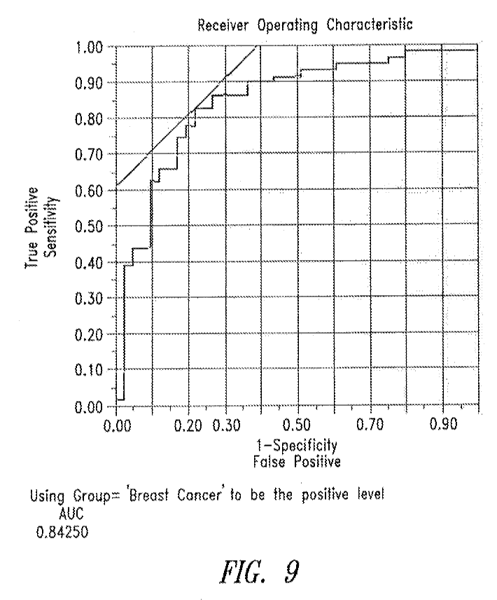

Nominal logistic regression analysis for breast cancer and control samples, was conducted using the two representative proteins. The generated ROC curve for S100A9 has an AUC of 0.78220 (FIG. 7), and LG3BP has an AUC of 0.76088 (FIG. 8). The analysis was repeated using two proteins and an AUC of 0.84250 was obtained (FIG. 9)

Sample population characteristics: Clinical data such as breast density, cancer type, and tumor size were obtained on as many samples as possible.

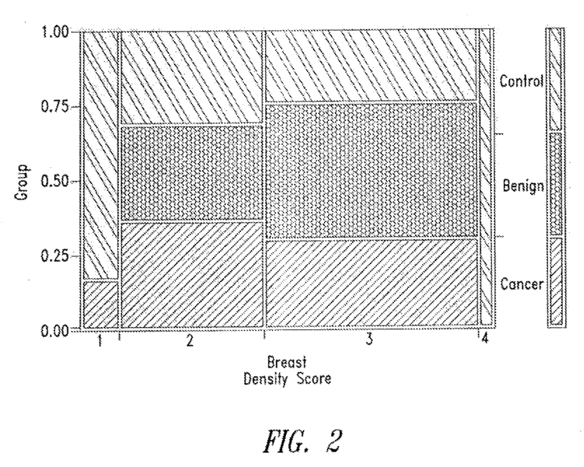

Experiment 1: Out of the 75 samples tested, breast density scores were obtained for 41 of the samples. The break down of the sample population by breast density was 14% category 1, 36.6% category 2, 43,9% category 3, 4.7% category 4 (FIG. 2). Eighteen of the twenty-five cancer samples were collected from patients diagnosed with Intra ductal carcinoma (IDC). Two patients were diagnosed with ductal carcinoma In Situ (DCIS), one patient was diagnosed with lobular carcinoma In Situ (LCIS), and one patient was diagnosed with intra lobular carcinoma (ILC).

For classification a scale was designed where a classification of “small” was given to tumors <20 mm, a classification of “medium” was assigned to tumors between 21 mm-99 mm, and a classification of “large” was assigned to tumors >1 cm. Seventeen of the samples collected from breast cancer patients were classified as large tumors, two samples were classified as medium, and six were classified as small. Pathology information of receptor type was obtained for eight of the cancer samples. One sample was ER−/HER2−, one sample was ER−/PR−, two samples were ER+/HER2−, three samples were ER+/HER2+, and one sample was ER+/PR−.

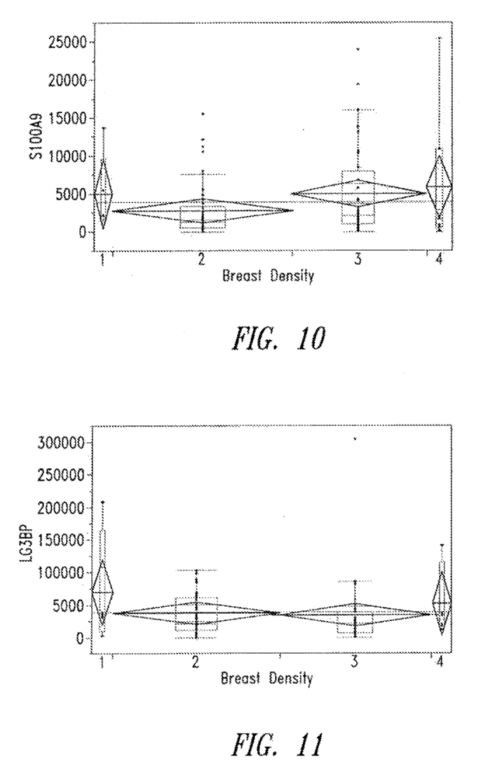

Experiment 2: The sample pool analyzed in this study consisted of 5.9% category 1 breast tissue; 50% category 2; 37.8% category 3, and 5.4% category 4. ANOVA analysis was conducted on several proteins evaluated by ELISA to determine if breast tissue type affects protein expression levels. Representative data is provided for two proteins studied (FIG. 10 and FIG. 11). Using an alpha level of 0.05, no statistically significant difference was observed for the means of the four types of breast tissue for each of the proteins evaluated in the ELISAs shown in FIGS. 10 and 11.

Samples were collected from a variety of breast cancer types resulting in 17 samples from patients diagnosed with DCIS, 44 samples from patients diagnosed with IDC, 4 samples from patients with both DCIS & IDC, and 5 samples from patients with ILC. Tumor size varied tremendously. Using that classification system described for Experiment 1, for the samples where tumor size was provided, 33 samples from patients with large tumors were tested, 9 samples from patients with medium tumor size, and 12 samples from patients with small tumor size. Of the 38 samples where receptor type was provided 1 sample was ER−, 2 samples were ER−/HER2−, 2 samples were triple negative (ER−/HER2−/PR−), 1 sample was ER−/HER2+, 11 samples were ER+, ER+/HER2 16, and 5 samples were ER+/HER2+. To date no distinguishing trend has been observed based on tumor size, cancer type, or receptor status, for the proteins that have been studied.

### REFERENCES

Armstrong, K., Handorf, E. A., Chen, J., & Bristol Demeter, M. N. (2013). Breast cancer risk prediction and mammography biopsy decisions: a model-based study. American Journal of Preventive Medicine, 44(1), 15-22, doi:10.1016/j.amepre.2012.10.002
Bigenwald, R. Z., Warner, E., Gunasekara, A., Hill, K. A., Causer, P. A., Messner, S. J., Eisen, A., Plewes, D. B., Narod, S. A., Zhang, L., Yaffe, M. J. (2008) Is Mammography Adequate for Screening Women with Inherited BRCA Mutations and Low Breast Density?. Cancer Epidemiology Biomarkers Prevention. 17(3); 707-711.
Böhm, D., Keller, K., Pieter, J., Boehm, N., Wolters, D., Siggelkow, W., et al. (2012). Comparison of tear protein levels in breast cancer patients and healthy controls using a de nova proteomic approach. Oncology Reports, 28(2), 429-438. doi:10.3892/or.2012.1849
Böhm, D., Keller, K., Wehrwein, N., Lebrecht, A., Schmidt, M., Kölbl, H., & Grus, F.-H. (2011). Serum proteome profiling of primary breast cancer indicates a specific biomarker profile. Oncology Reports, 26(5), 1051-1056. doi:10.3892/or.2011.1420
Boyd, N. F, Guo, H., Martin, L. J., Sun, L., Stone, J., Fishell, E., Song, R. A., Hislop, G., Chiarelli, A., Minkin, S., Yaffee, M. J., (2007) Mammographic Density and the Risk and Detection of Breast Cancer. The New England Journal of Medicine. 356(3): 227-236).
Brown, M. L., Houn, F., Sickles, E. A., & Kessler, L. G. (1995). Screening Mammography in Community Practice: Positive Predictive. American Journal of Radiology, 165, 1373-1377.
CLIA Laws and Regulations, 2013, http://wwwn.cdc.gov/CLIA/Regulatory/default.aspx. Grady, D. (2012). Study of Breast Biopsies Finds Surgery Used Too Extensively, New York Times, 1-4.
Klifa, C., Carballido-Gamio, J., Wilmes, L., Laprie, A., Shepherd, J., Gibbs, J., Fan, B., Noworolski, S., Hylton, N. (2010) Magnetic resonance imaging for secondary assessment of breast density in a high-risk cohort. Magnetic Resonance Imaging. 28; 8-15.
Kolb, T., Lichy, J., & Newhouse, J. (2002). Comparison of the performance of screening mammography, physical examination, and breast US and evaluation of factors that influence them: an analysis of 27,825 patient evaluations. Radiology, 225(1), 165-175.
Lebrecht, A., Boehm, D., Schmidt, M., Koelbl, H., & Gnus, F. H. (2009a). Surface-enhanced Laser Desorption/Ionisation Time-of-flight Mass Spectrometry to Detect Breast Cancer Markers in Tears and Serum. Cancer Genomics & Proteomics, 6(2), 75-83.
Lebrecht, A., Boehm, D., Schmidt, M., Koelbl, H., Schwirz, R. L., & Grus, F. H. (2009b). Diagnosis of breast cancer by tear proteomic pattern. Cancer Genomics & Proteomics, 6(3), 177-182.
Li, J., Zhang, Z., Rosenzweig, J., Wang, Y., & Chan, D. (2002). Proteomics and bioinformatics approaches for identification of serum biomarkers to detect breast cancer. Clin Chem, 48(8), 1296-1304.
Luftner, D., & Possinger, K. (2002). Nuclear matrix proteins as biomarkers for breast cancer. Expert Rev Mol Diagn, 2(1), 23-31. doi:ERM020106 [pii]
10.1586/14737159.2.1.23
Pisano, E. D., Gatsonis, C., Hendrick E., Yaffe, M., Baum, J. K., Acharyya, S., Conant, E. F. Fajardo, L. L., Bassett, L., D'Orsi, C. Jong, R., Rebner, M. (2005). Diagnostic Performance of Digital versus Film Mammography for Breast-Cancer Screening. The New England Journal of Medicine. 17(353). 1773-1783.
Scheel, J. R., Lee, J. M., Sprague, B. L., Lee, C. I., Lehman, C. D. (2015) Screening ultrasound as an adjunct to mammography in women with mammographically dense breasts. Gynecology. 212(1); 9-17.
Schiess, R., Wollscheid, B., & Aebersold, R. (2009). Targeted proteomic strategy for clinical biomarker discovery. Molecular Oncology, 3(1), 33-44. doi:10.1016/j.molonc.2008.12.001
Tabar, L., Vitak, B., Chen, T. H., Yen, A. M., Cohen, A., Tot, T., Chiu, S., Chen, S. Fann, J. Rosell, J., Fohlin, H., Smith, R. A., Duffy, S. W., (2011) Swedish Two-County Trial: Impact of Mammographic Screening on Breast Cancer Mortality during 3 Decades. Radiology, 3(260), 658-663.
Vachon, C. M., van Gils, C. H., Sellers, T. A., Ghosh, K., Pruthi, S., Brandt, K. R., Pankratz, V. S., (2007) Mammographic density, breast cancer risk and risk prediction. Breast Cancer Research 9:2017 (doi:10.1186/bcr1829).
Wu, K., & Zhang, Y. (2007). Clinical application of tear proteomics: Present and future prospects. Proteomics. Clinical Applications, 1(9), 972-982. doi:10.1002/prca.200700125

1-22. (canceled)

23. An immunoassay, comprising
(a) a lacrimal secretion sample from a human subject, wherein the subject is suspected of having breast cancer, or has a palpable lump suspected of being cancerous, wherein the lacrimal secretion sample comprises at least two protein biomarkers comprising S100A9 and LG3BP; and
(b) binding agents and reagents that accommodate a binding interaction specific to the at least two protein biomarkers in (a) to produce a reaction which can be quantitated based on production of a detectable signal,
wherein the levels of S100A9 are increased relative to a non-cancer control sample, and wherein the levels of LG3BP are decreased relative to the non-cancer control sample.

24. The immunoassay of claim 23, wherein the lacrimal secretion sample comprises at least three protein biomarkers comprising S100A9, LG3BP, and S100A8, and wherein the binding agents comprise antibodies that specifically bind to the S100A9, LG3BP, and S100A8 protein biomarkers.

25. The immunoassay of claim 24, wherein wherein the levels of S100A9 and S100A8 are increased relative to a non-cancer control sample, and wherein the levels of LG3BP are decreased relative to the non-cancer control sample.

26. The immunoassay of claim 23, wherein the lacrimal secretion sample is an ocular wash sample.

27. The method of claim 23, wherein the human subject has Stage I cancer or Stage II cancer, or a later stage

28. The immunoassay of claim 23, wherein the breast density of the subject is category 1, category 2, category 3, or category 4.
